(19) United States
(12) Patent Application Publication
(10) Pub. No.: US 2023/0389960 A1
(43) Pub. Date: Dec. 7, 2023

(54) EXPANDABLE PNEUMO-OCCLUDER WITH SEALABLE INSTRUMENT PORT

(71) Applicant: Intelligent Surgical Devices LLC, Bloomfield, NY (US)

(72) Inventors: Todd Michael Haran, Bloomfield, NY (US); Madonna Tomani, Farmington, NY (US)

(73) Assignee: Intelligent Surgical Devices LLC, Bloomfield, NY (US)

(21) Appl. No.: 17/833,250

(22) Filed: Jun. 6, 2022

(51) Int. Cl. A61B 17/42

(52) U.S. Cl. CPC A61B 17/4241; A61B 2017/4216; A61B 2017/00557

(57) ABSTRACT

A surgical apparatus for insertion into an annular orifice of an animal body (e.g., vaginal orifice) to provide cavity access to a cavity while maintaining pneumoperitoneum that includes an expandable member, like an outer bladder or balloon like structure (similar to a pessary), surrounding a central structural member, that typically would be ring shaped and a flexible opening or penetrable port, similar to an elastomeric duck bill valve, affixed within an interior of said central structural member, through which an instrument or device may be inserted to provide surgical access to the cavity. The penetrable port may be of an active or passive configuration where, in the active configuration, it can be expanded to conform to the instruments. In the passive form, the penetrable port is comprised of an elastomeric or similar material that is fixed in shape and conformably flexes and seals to the instrument maintaining pneumoperitoneum.

### FIELD OF THE INVENTION

A surgical apparatus that provides instrument access to a procedure's inherent or created cavity while maintaining pressurization of the cavity is disclosed. The cavity can be an annular orifice of an animal body (e.g., vaginal orifice) where the invention provides the procedure's instrument access via a central ring while maintaining cavity inflation (pneumoperitoneum) via an expandable outer surface, bladder or balloon like-structure, surrounding the central ring.

### BACKGROUND OF INVENTION

Most hysterectomies are performed under pneumoperitoneum where the abdominal cavity is inflated, creating space between internal organs and tissues, making it easier for the surgeon to perform the procedure. Similar to many other abdominal procedures, a hysterectomy is performed laparoscopically through the abdominal wall. In these procedures, trocars are used to maintain the pneumoperitoneum while acting as a portal for surgical instruments through the abdominal wall into the abdominal cavity.

A trocar is pneumo-occluder that is inserted through the abdominal wall. It typically includes a cannula and has an airtight port at its center. The port maintains an airtight seal beyond the trocar into the cavity as graspers, scissors, staplers, cameras and other surgical instruments may pass through the port to access the abdominal cavity during procedures.

Hysterectomies pose a unique challenge that is not seen in other abdominal procedures. In most other procedures, the abdominal tissue inherently creates an airtight chamber that in effect maintains pneumoperitoneum until the procedure is complete. In those cases, the typical trocar works effectively. However, during a hysterectomy the uterus is cut from the inner portion of the vagina in what is known as a colpotomy or vaginotomy. When the colpotomy is performed, pneumoperitoneum is lost through the incision and out through the vagina with the following affects: without sufficient pneumoperitoneum, the space between internal organs and tissues begins to close-in making it difficult for the surgeon to complete the procedure; as air escapes out through the colpotomy incision, it pushes bodily fluids with it which inhibits the procedure and exposes the incision to infection; the procedure time is increased with more potential follow-up, and may, therefore, be more expensive with increased medical risk; there is an increased opportunity for error, such as in the sponge count; risks of bowel injury; and increased anesthesia exposure.

U.S. Pat. No. 8,287,552 (the '552 Patent) discloses a surgical device for accessing a hollow cavity via an elongated body including an outer sealing member engaging an inner surface of the passage and a working channel or port extending therethrough into the hollow cavity. A plurality of suction holes formed through an outer wall of the elongated body provides a vacuum channel for increasing the sealing force between the tissue of the entrance passage to the outer surface of the elongated body. While the '552 Patent provides for a negative pressure to seal the cavity to the organ, the passage of instruments through the device appears to be taught by reference to the working channel “to permit therapeutic fluids and/or instruments to be inserted into the hollow organ therethrough.” '552 Patent, Col. 2, line 45-47. The working channel preferably extends substantially along a longitudinal axis of the sealing member, to provide a passage into the uterus.

U.S. Patent Publication No. 2013/0345714 discloses a uterine manipulator having an elongate shaft and a tip hub. The distal end is inserted into a vagina. The tip hub is disposed at the distal end and is configured to releasably receive and support a tip mount for engaging a uterus. The elongate shaft includes one or more channels to receive catheter tubing. In one embodiment, a vaginal occluder includes a main body having an expandable balloon cuff, wherein a balloon cuff tube inflates the cuff when desired. The openings allow the catheter tubes to be inserted into and removed from the channels. The channel can have sufficient depth such that the catheter tubes lie flush with the outer surface of the shaft. This can facilitate devices, such as other medical instrumentation, to be advanced, e.g., coaxially, along the shaft.

U.S. Pat. No. 5,840,077 discloses a colpotomy assembly for insertion into a vaginal cavity for use in female pelvic surgical procedures employing the inflation of an abdominal cavity with a gas to facilitate the accessibility to, and visibility of, female pelvic organs. The assembly has a vaginal extender, a uterine manipulator and a flexible occluder. The occluder is mounted on a shaft of the uterine manipulator such that it permits relative movement of the shaft during use.

U.S. Patent Publication No. 2015/0005780 discloses a uterine manipulator comprising a pneumo-occluder that enables rotation of the manipulator shaft while maintaining pneumoperitoneum. The pneumo-occluder can be positioned within the vagina when the uterine manipulator is set within the uterus. Once in position, the balloon can be inflated via the filling tube in order to seal the distal vaginal cavity from the proximal vaginal cavity.

U.S. Patent Publication No. US 2013/0023896 (the '896 Publication) provides for uterine manipulators having a vaginal occlusion portion comprising a resilient deformable member including a channel having an opening sized to fit against a manipulation rod, while minimizing movement of the vaginal occlusion member when the rod is manipulated, and wherein the member is configured to fit against the contours of an inner vaginal tissue surface. Also provided are surgical methods using the device to provide for a snug fit with the cervix, while sealing the vagina, to maintain abdominal insufflation during surgery.

Notably, Paragraph [0081] of the '896 Publication states:
[p]referably, the resilient vaginal occluder/sealer element comprises an axial channel having a small opening at the proximal end for the tube to slip into and a larger opening (or a smaller distal opening subtended by a deformable material; to provide for a distal deformable grommet opening), at the distal end that allows for a tube or catheter to be inserted into the small proximal opening through the channel and out a larger distal opening (or out the smaller distal opening subtended by a deformable material; to provide for a distal deformable grommet opening). The large distal opening (or the smaller distal opening subtended by a deformable material; to provide for a distal deformable grommet opening) is configured to allow significant angular and/or rotational manipulation/adjustment of the tubing, or semi-rigid rod surrounding the tubing, without allowing leakage of the gas used to insufflate the abdomen. One of skill in the art will, based on the present teachings, recognize that other shapes having these general characteristics and formed of suitable material may be used in practicing the invention.

U.S. Pat. No. 5,643,285 (the '285 Patent) includes an occluder which is inflated to seal the distal vaginal cavity from the proximal vaginal cavity. The vaginal occluder prevents the escape of gas used to inflate the abdominal cavity. See '285 Patent, Col. 4, line 7 (“Occluder may also be provided with an access port and is configured to permit the insertion of surgical tools therethrough.”)(reference numerals omitted).

### SUMMARY OF THE INVENTION

An exemplary aspect of the present invention may be directed to a pneumo-occluder with an expandable sealing port at its center. In one aspect, the pneumo-occluder is specifically designed to fit into the vagina of a patient, forming a seal that is capable of maintaining pneumoperitoneum during and after the colpotomy is performed. The flexible sealed port maintains pneumoperitoneum while instruments are passed through the port and the severed reproductive organs may be removed through it.

In a further aspect of the present invention, non-limiting examples of a pneumo-occluder may include one or more of the following features: an expanding outer diameter that actively seals against the walls of the vagina; a flexible outer construction allowing the outer diameter (OD) of the occluder to be variable and controllable by inflation; a flexible port, having a contracting inner diameter (ID) that actively seals against instruments when inserted through the occluder port; a flexible outer OD construction expansion potential (e.g., 2″-4″); a port configured to be compatible with camera, forceps, scalpel, suction devices, graspers, suture devices, surgical instruments and other common procedure tools; and inflation/deflation devices.

In another aspect, the present invention includes a surgical apparatus for insertion into an annular orifice of an animal body to provide access to a cavity defined by the animal body while the surgical apparatus creates a seal within the annular orifice to maintain pneumoperitoneum during a surgical procedure. The surgical apparatus comprising a central support member, an outer expandable member, and a flexible opening member. The outer expandable member is coupled to the central support member and is configured to expand to create a seal with the annular orifice. The flexible opening member is disposed within the central support member and has a first state and a second state. In the first state, the flexible opening member is normally closed or sealed to prevent fluidic communication between opposing sides of the flexible opening member. In the second state, a device or an instrument is passed through the flexible opening member and the flexible opening member conforms and seals against the device or the instrument to prevent fluidic communication between opposing sides of the flexible opening.

In another aspect, the outer expandable member may be adjustably expandable to control the contact and seal with the annular orifice to resist the flow of fluid between the cavity and an ambient operating room pressure. The outer expandable member may be configured to be inflatable using an external inflation device, wherein the external inflation device includes a pressurization source for providing a fluid volume within a cavity defined by the outer expandable member. The fluid volume may be provided manually or automatically by the pressurization source which expands the outer expandable member to maintain the seal with the annular orifice. The apparatus may further include a valve that is configured to selectively maintain the fluid volume within the outer expandable member and maintain the seal between the annual orifice and the outer expandable member.

In yet another aspect, a first side of the apparatus faces a surgical procedure within the cavity, and a second side of the apparatus faces a surgeon using the apparatus. The apparatus isolates the cavity pneumoperitoneum pressure from ambient atmospheric pressure. When the surgeon inserts the device or the instrument through the flexible opening member into the cavity, the flexible opening member expands by the insertion of the device or the instrument allowing the passage of the device or the instrument into the cavity while sealing against an outer surface of the device or the instrument which allows for maintaining pneumoperitoneum pressure. The flexible opening member and the device or the instrument may both have outer surfaces configured to ensure the seal between the flexible opening member and the device or the instrument. In the first state, the flexible opening member may be a normally collapsed aperture, and in the second state, the aperture is expandable to permit non-permanent displacement and penetration by, yet circumferentially sealed about, the outer surface of the device or the instrument.

In another aspect, the central support member, the outer expandable member, and the flexible opening member may be singularly constructed out of a single material using a process such as casting or molding, wherein the central support member is at least twice the thickness of either the outer expandable member or the flexible opening member, and wherein the central support member will resist deformation when the outer expandable member is expanded and/or the flexible opening member is moved from the first state to the second state. The outer expandable member may be expanded by inflating a cavity defined by the outer expandable member with an expansion fluid, wherein the expansion fluid is supplied by an external pressure generator through a tube connected to the outer expandable member.

In yet another aspect, the outer expandable member may originally be a separate piece and attached to the central support member. Further, the central support member has an opening defined therein, wherein the flexible opening member is a separate piece relative to the central support member and is attached to the opening of the central support member. The opening of the central support member may be circular and have a diameter less than 50 millimeters.

Disclosed in embodiments herein is a surgical apparatus for insertion into an annular orifice of an animal body to provide cavity access while maintaining pneumoperitoneum during a surgical procedure. The surgical apparatus may comprise an expandable outer bladder or balloon-like structure surrounding a central region ring and a penetrable port affixed within an interior of the central region ring, through which an instrument or device may be inserted to provide surgical access to the cavity.

### DESCRIPTION OF THE DRAWINGS

FIG. 1 is an illustration of the reproduction organs of a woman, with an expanded view of an exemplary embodiment of a surgical apparatus in accordance with an aspect of the present invention positioned within the vagina;

FIG. 2, is an illustration of the exemplary surgical apparatus shown in FIG. 1 within the vagina in a deflated condition;

FIG. 3 is an enlarged illustration of the exemplary surgical apparatus shown in FIG. 1 within the vagina in an inflated condition;

FIG. 4 is a top plan view of the exemplary surgical apparatus shown in FIG. 1;

FIG. 5A shows an alternative exemplary embodiment of a surgical apparatus having an inflatable sealing port; and

FIG. 5B shows another alternative exemplary embodiment of surgical apparatus having an alternative inflatable sealing port.

### DETAILED DESCRIPTION OF THE INVENTION

As noted above, hysterectomies pose a unique challenge not present in other abdominal surgery. In other abdominal procedures the abdominal tissue inherently seals against the instrument creating an airtight chamber or envelope that effectively maintains pneumoperitoneum until the procedure is complete. In such cases a conventional trocar works effectively. During a hysterectomy, however, the uterus is cut from the inner portion of the vagina in what is known as a colpotomy (or vaginotomy). When the colpotomy is performed, pneumoperitoneum is lost through the incision and through the vagina. Without sufficient pneumoperitoneum, space between internal organs and tissues begins to close contributing to aforementioned risks which cause difficulty for a surgeon to complete the procedure.

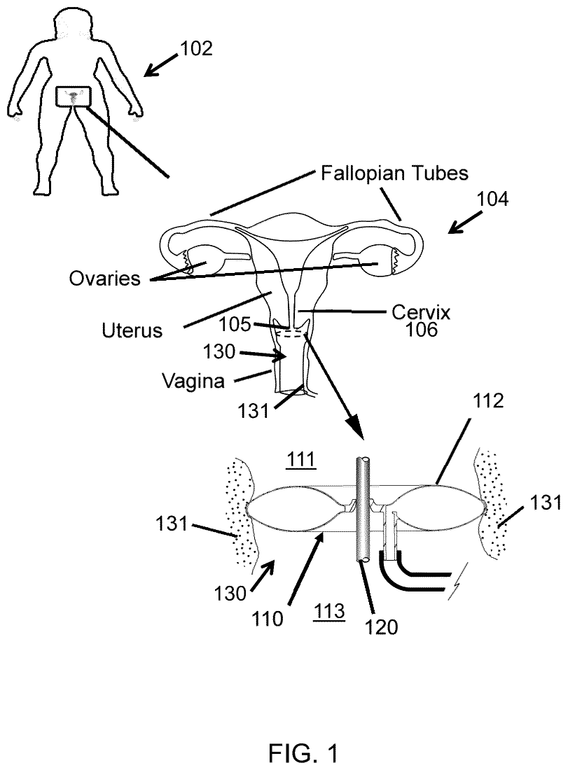

Referring now to FIG. 1, an exemplary embodiment of a pneumo-occluder apparatus in accordance with an aspect of the present invention is generally referenced as numeral 110. Pneumo-occluder 110 addresses the aforementioned problems by being configured for insertion into the annular vaginal orifice 130 of an animal body, such as a human 102, to provide procedural cavity access while maintaining pneumoperitoneum during a medical procedure, such as a hysterectomy which involves partial or complete removal of the female reproductive organs 104. FIG. 1 shows an exemplary use of pneumo-occluder apparatus 110 where it is placed into the vaginal orifice 130 wherein bladder 112 is inflated such that pneumo-occluder 110 engages and seals against the inner vaginal wall 131 defining vaginal orifice 130, as will be discussed in greater detail below.

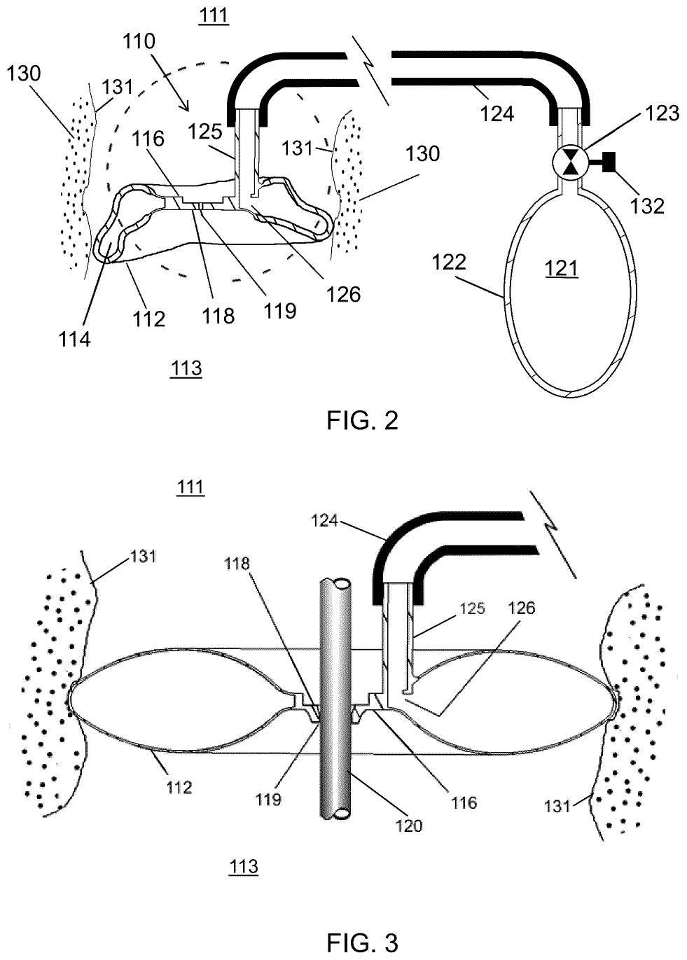

Turning now to FIG. 2, exemplary embodiment pneumo-occluder 110 is inserted configured to be inserted into vaginal orifice 131 with bladder 112 in a deflated condition. Pneumo-occluder 110 is advanced within vaginal orifice 131 until placed within the desired location, such as for example, proximate to external os 105 (FIG. 1) of cervix 106. As seen in FIG. 2, functional components of pneumo-occluder 110 may include, by example and without limitation thereto, bladder 112 defining a plenum 114, a base structure 116 including a penetrable sealing port 118 (i.e., flexible opening member) defining an aperture 119 therethrough, and a connection tube 125 fluidly coupled to plenum 114 via junction 126. Connection tube 125 may be coupled to an external pressurization source, such as but not limited to blood pressure inflation bulb 122, via a supply tube 124. It should be noted that, while pneumo-occluder 110 is shown combined into a singular body, alternative embodiments may include a pneumo-occluder constructed out of individual elements and joined to form a completed device.

With continued reference to FIG. 2, pneumo-occluder 110 is shown in its deflated/vaginal insertion state and pre-instrument insertion state. In use, such as during a hysterectomy procedure, the deflated pneumo-occluder 110 and coupled supply tube 124 are inserted into the vaginal orifice 130 of patient 102. Once positioned, bulb 122 is squeezed to thereby provide pressurized air to plenum 114 of bladder 112. Pressurized air is provided to plenum 114 until a seal is made between bladder 112 and vagina inner wall 131 which defines vaginal orifice 130 (see FIG. 3). Delivery of pressurized air via bulb 122 may be controlled using a valve 123 and associated valve knob 132.

Bulb valve 123 allows the compressed air to communicate between the bulb interior 121 and plenum 114 of bladder 112. Bulb 122 naturally expands after squeezing to thereby refill bulb 122 with ambient air 111 passing through valve 123. Bladder 112 remains pressurized until the air released from plenum 114 by manually opening manual vent screw 132, which is typically done after the surgical procedure is completed. While shown and described as a blood pressure inflation valve, it should be noted that any suitable alternate source of inflation may replace bulb 122, such as but not limited to a number of industry known devices including regulated pressurized gas commonly available in operating rooms.

FIG. 3 shows bladder 112 in the inflated state creating a barrier seal between the ambient air 111 (via vaginal orifice 130) and the internal cavity 113 within patient 102, such as the uterine cavity. As a result, pneumoperitoneum within internal cavity 113 may be maintained under until the procedure is complete. Furthermore, as shown in FIG. 3, an instrument 120 may be inserted into internal cavity 113 through aperture 119 of penetrable sealing port 118. Aperture 119 may conform to the outer surface of instrument 120, thereby creating a barrier seal between the ambient air 111 and internal cavity 113.

In one embodiment, as shown in FIG. 3, the components of pneumo-occluder 110 may be integrated into a single moldable unit which contains the key components (112, 116, 118, 119, 125 and 126) of the apparatus. In one aspect of the present invention, the walls of bladder 112 are substantially thinner (e.g., at least 2×) than the thickness of penetrable sealing port 118. Thus, upon pressurization of bladder 112 when placed within the annular orifice 131 of the vagina, penetrable sealing port 118 resists deformation as bladder 112 expands.

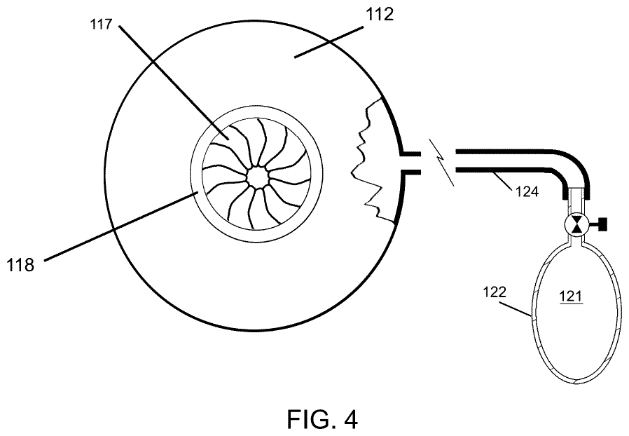

With additional reference to FIG. 4, pneumo-occluder 110 may comprise outer expandable member or outer bladder 112, such as an iris, expandable bladder or inflatable balloon-like structure, which surrounds rigid member or central ring-shaped base support structure 116. Support structure 116 may be mounted or sealed to bladder 112. Penetrable sealing port 118 may be constructed of a compliant (elastomeric) air sealing gasket 117 defining aperture 119, such as a slit or through-hole, therein that allows passage of one or more instruments 120. For example, aperture may be circular and have a diameter less than 50 millimeters. As each instrument 120 penetrates aperture 119, gasket 117 stretches while providing an airtight seal around the instrument 120, thereby preventing leaks from communicating between ambient pressure 111 and cavity pressure 113 as described above.

In another embodiment, aperture 119 may be defined by a material which is non-homogeneous with the remainder of penetrable sealing port 118. For example, aperture 119 may be formed within a material that is co-molded or insert molded with the remainder of penetrable sealing port 118. One non-limiting example may be similar to a duck bill valve configuration molded onto the support structure of the penetrable sealing port 118.

Examples of the type of materials and configuration of a passive sealing port are those that may be described as a puncturable sealing port employed to maintain pneumoperitoneum (e.g., U.S. Pat. No. 4,746,023). Other examples of materials and techniques to effect a passive seal about a surgical device inserted through the passive seal include not only self-sealing or conformable elastomeric “valves” (e.g., duckbill, U.S. Pat. Nos. 5,330,437; 5,350,364; 8,147,457) used to seal trocar-type or colpotomy devices, but also seals disclosed in the following U.S. Pat. Nos. 5,158,553, 5,554,124. All of the foregoing being hereby incorporated by reference in their entirety.

And, in yet another alternative embodiment, penetrable sealing port 118 may be a non-homogenous part of inflatable pessary that is assembled, for example inserted and/or attached, into bladder 112 after molding. Such an embodiment would permit the inclusion of dissimilar materials for pneumo-occluder 110, and are attached to one another.

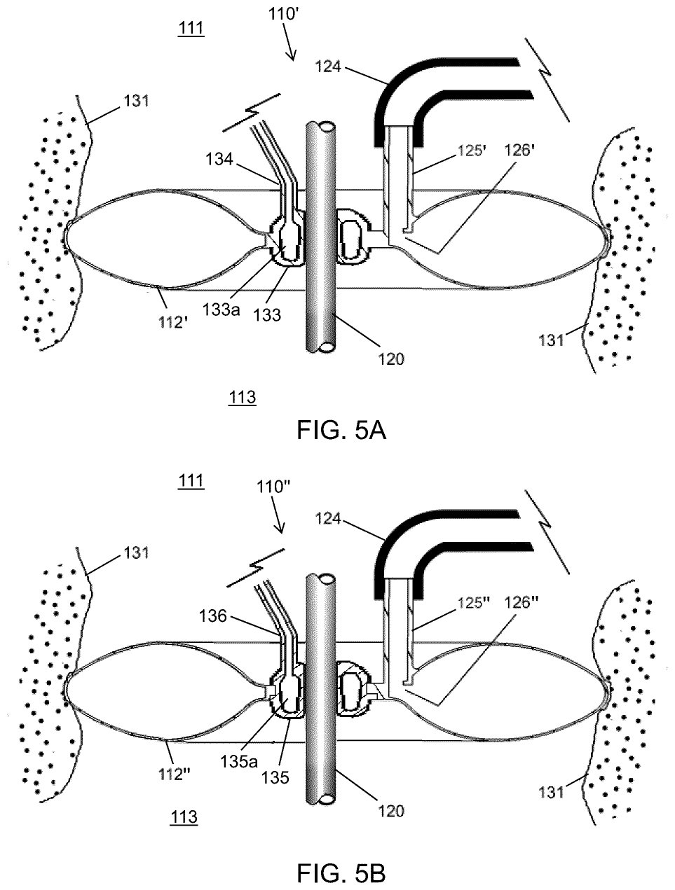

In an alternative embodiment, for example as depicted in FIG. 5A, pneumo-occluder 110′ may be constructed out of a flexible material that includes an inflatable sealing port 133 defining a separate cavity 133a that can be inflated independently from the bladder 112′ via an inflatable sealing port tube 134 while bladder 112′ is inflatable via connection tube 125′ and junction 126′ through supply tube 124 as described above with regard to pneumo-occluder 110.

FIG. 5B shows another alternative embodiment of a pneumo-occluder 110″ where the inflatable sealing port 135 is non-homogeneous to the bladder 112″ and defines a separate cavity 135a that can be inflated independently from bladder 112″ via inflatable sealing port tube 136 while bladder 112″ is inflatable via connection tube 125″ and junction 126″ through supply tube 124. Non-limiting examples include the inflatable sealing port 135 being co-molded or over-molded with bladder 112. Inflatable sealing port 135 may also be constructed of a different material than the remainder of pneumo-occluder 110″. In another non-limiting alternative, inflatable sealing port 135 may also be made of a separate, non-homogeneous piece than bladder 112″ which is then physically connected to bladder 112″ before use.

The inflatable sealing ports 133, 135 described above may be generally referred to as active sealing ports.

Although not forming part of the disclosed apparatus or embodiments, the following list of surgical instruments and devices are contemplated as being suitable for use with one or more of the penetrable/sealing port embodiments: Tenaculum (e.g., Single Tooth, Double Tooth, etc.) Graspers (e.g., Kelly, Babcock, Allis, etc.), Bags (e.g., Endobags), Hysteroscope and other cameras, Ring Forcep, Biopsy instrument(s), any Laparoscopic instrument(s), Speculum and VCare.

It should also be noted that the intention of the sealing portions of the apparatus and body tissue may result in leaks. The ability to offset any small leaks can be accomplished any number of ways, such as re-inflation of the bladder via the pressurization bulb, or in the case of operating room regulated air pressure, that the regulated pressure maintains inflation automatically.

In summary and without limitation thereto, the integrated design of the pessary-like inflatable bladder encompasses one of two sealing port configurations: the passive/penetrable sealing port, and the inflatable sealing port. As described above, the passive approach employs one or more passive sealing ports through which implements, and tissues can be passed while maintaining pneumoperitoneum. Similarly, the active approach also employs one or more active seals through with implements and tissues can be passed while maintaining pneumoperitoneum, where the active port is an inflatable/deflatable independent of the pessary.

It should be understood that various changes and modifications to the embodiments described herein will be apparent to those skilled in the art. Such changes and modifications can be made without departing from the spirit and scope of the present disclosure and without diminishing its intended advantages. It is therefore anticipated that all such changes and modifications be covered by the instant application.

What is claimed is:

1. A surgical apparatus for insertion into an annular orifice of an animal body to provide access to a cavity defined by the animal body while the surgical apparatus creates a seal within the annular orifice to maintain pneumoperitoneum during a surgical procedure, the surgical apparatus comprising:
a) a central support member;
b) an outer expandable member that coupled to the central support member, wherein the outer expandable member is configured to expand to create a seal with the annular orifice;
c) a flexible opening member disposed within the central support member, wherein the flexible opening member has a first state and a second state,
wherein in the first state, the flexible opening member is normally closed or sealed to prevent fluidic communication between opposing sides of the flexible opening member, and
wherein in the second state, a device or an instrument is passed through the flexible opening member and the flexible opening member conforms and seals against the device or the instrument to prevent fluidic communication between opposing sides of the flexible opening.

2. The apparatus according to claim 1 wherein the outer expandable member is adjustably expandable to control the contact and seal with the annular orifice to resist the flow of fluid between the cavity and an ambient operating room pressure.

3. The apparatus according to claim 2 wherein the outer expandable member is configured to be inflatable using an external inflation device.

4. The apparatus according to claim 3, wherein the external inflation devices includes a pressurization source for providing a fluid volume within a cavity defined by the outer expandable member, wherein the fluid volume is provided manually or automatically by the pressurization source which expands the outer expandable member to maintain the seal with the annular orifice.

5. The apparatus according to claim 4 further comprising a valve, wherein the valve is configured to selectively maintain the fluid volume within the outer expandable member and maintain the seal between the annual orifice and the outer expandable member.

6. The apparatus according to claim 1 wherein a first side of the apparatus faces a surgical procedure within the cavity, wherein a second side of the apparatus faces a surgeon using the apparatus, wherein the apparatus isolates the cavity pneumoperitoneum pressure from ambient atmospheric pressure, wherein the surgeon inserts the device or the instrument through the flexible opening member into the cavity, where the flexible opening member expands by the insertion of the device or the instrument allowing the passage of the device or the instrument into the cavity while sealing against an outer surface of the device or the instrument which allows for maintaining pneumoperitoneum pressure.

7. The apparatus according to claim 1 wherein the flexible opening member and the device or the instrument both have outer surfaces configured to ensure the seal between the flexible opening member and the device or the instrument.

8. The apparatus according to claim 7, wherein said flexible opening member is a normally collapsed aperture in the first state, and wherein in the second state the aperture is expandable to permit non-permanent displacement and penetration by, yet circumferentially sealed about, the outer surface of the device or the instrument.

9. The apparatus according to claim 1, wherein the central support member, the outer expandable member, and the flexible opening member are singularly constructed out of a single material using a process such as casting or molding, wherein the central support member is at least twice the thickness of either the outer expandable member or the flexible opening member, and wherein the central support member will resist deformation when the outer expandable member is expanded and/or the flexible opening member is moved from the first state to the second state.

10. The apparatus according to claim 9 wherein the outer expandable member is expanded by inflating a cavity defined by the outer expandable member with an expansion fluid, wherein the expansion fluid is supplied by an external pressure generator through a tube connected to the outer expandable member.

11. The apparatus according to claim 1 wherein the outer expandable member originally is a separate piece and is attached to the central support member.

12. The apparatus according to claim 1 wherein the central support member has an opening defined therein, wherein the flexible opening member is a separate piece relative to the central support member and is attached to the opening of the central support member.

13. The apparatus according to claim 12 wherein opening of the central support member is circular and has a diameter less than 50 millimeters.

14. The apparatus according to claim 1 wherein the outer expandable member is mounted to the central support member.

15. The apparatus according to claim 14 wherein the outer expandable member is sealed to the central support member.
